(19) United States
(12) Patent Application Publication
(10) Pub. No.: US 2022/0064328 A1
(43) Pub. Date: Mar. 3, 2022

(54) FORMULATION OF NANOANTIBODY BASED DRUGS AND A METHOD FOR TREATING THROMBOTIC THROMBOCYTOPENIC PURPURA BY INHALATION

(71) Applicant: Anovent Pharmaceutical (U.S.), LLC, Newark, NJ (US)

(72) Inventors: Cai Gu Huang, Shrewsbury, MA (US); Hitesh Bhagavanbhai Mangukiya, Shanghai (CN)

(21) Appl. No.: 17/464,789

(22) Filed: Sep. 2, 2021

Related U.S. Application Data
(60) Provisional application No. 63/073,889, filed on Sep. 2, 2020.

(51) Int. Cl. C07K 16/36; A61K 47/54; A61K 47/52; A61K 9/00; A61K 9/08

(52) U.S. Cl. CPC C07K 16/36; A61K 47/549; A61K 47/542; C07K 2317/569; A61K 9/0078; A61K 9/08; A61K 47/52

(57) ABSTRACT

The present invention relates to stable formulations of a solution of nanoantibody drug that is suitable for inhalation and a method of treating acquired thrombotic thrombocytopenic purpura by administering the drug by inhalation using a soft mist inhaler or nebulizer. The pharmaceutical formulation for inhalation comprises caplacizumab.

### PRIORITY STATEMENT

This application claims the benefit of the filing date of U.S. Provisional Patent Application No. 63/073,889, filed on Sep. 2, 2020, which is incorporated herein by reference in its entirety.

### BACKGROUND OF THE INVENTION

Caplacizumab is a von Willebrand factor (vWF)-directed antibody fragment that targets the A1-domain of vWF, and inhibits the interaction between vWF and platelets, thereby reducing both vWF-mediated platelet adhesion and platelet consumption. Caplacizumab is a vWF-directed antibody fragment that consists of two identical humanized building blocks, linked by a three-alanine linker. Caplacizumab is produced in Escherichia coli by recombinant DNA technology and has an approximate molecular weight of 28 kDa. Caplacizumab is marketed by Ablynx as CABLIVI (caplacizumab-yhdp).

Acquired thrombotic thrombocytopenic purpura (aTTP or acquired TTP) is a rare blood disorder with an incidence of about three cases per million adults per year. Acquired TTP is a thrombotic microangiopathy, a disease of excessive blood clotting in small vessels throughout the body. The clots can limit or block the flow of oxygen-rich blood to the body's organs, such as the brain, kidneys, and heart. Acquired TTP is caused by inhibitory autoantibodies against ADAMTS13, a protease that cleaves von Willebrand factor. When von Willebrand factor is not cleaved, aberrant coagulation produces small-vessel platelet-rich thrombi that cause consumptive thrombocytopenia, microangiopathic hemolytic anemia, bleeding, and end organ damage. As a result, serious health problems can develop. The increased clotting that occurs in TTP also uses up platelets in the blood. Platelets are blood cell fragments that help form blood clots. These cell fragments stick together to seal small cuts and breaks on blood vessel walls and stop bleeding. With fewer platelets available in the blood, bleeding problems can occur. People who have TTP may bleed inside their bodies, underneath the skin, or from the surface of the skin. When cut or injured, they also may bleed longer than normal. A lack of activity in the ADAMTS13 enzyme (a type of protein in the blood) causes TTP. The ADAMTS13 gene controls the enzyme, which is involved in blood clotting. The enzyme breaks up a large protein called von Willebrand factor that clumps together with platelets to form blood clots.

TTP usually occurs suddenly and lasts for days or weeks, but it can continue for months. Relapses (or flareups) can occur in up to 60 percent of people who have the acquired type of TTP. Many people who have inherited TTP have frequent flareups that need to be treated. In clinical practice, the treatment for aTTP includes daily fresh frozen plasma for people who have inherited TTP or plasma exchange therapy for people who have acquired TTP until the patient's platelet counts have returned to baseline and there is no further evidence of microangiopathic hemolytic anemia and end organ damage. For patients who do not respond to daily plasma exchange, or for those whose initial disease presentation is considered severe, that is when ADAMTS13 activity levels remain below 10%, corticosteroids are generally added to the daily plasma exchange.

Other treatments are used if plasma therapy does not work well or if flareups occur often. Other treatments include off-label use of rituximab and, sometimes, additional immunosuppressive agents such as cyclophosphamide, vincristine, or cyclosporine. A high proportion of patients, anywhere from 15-20%, have a recurrence of the disease when plasma exchange is stopped. Medications can be taken in a variety of ways, such as by swallowing, by inhalation, by absorption through the skin, or by intravenous injection. Each method has advantages and disadvantages, and not all methods can be used for every medication. Improving current delivery methods or designing new ones can enhance the efficacy and use of existing medications to expand the clinical benefit to a broader patient population and to improve outcomes in the treatment of TTP.

As part of the Biologics Price Competition and Innovation Act (BPCIA), a biological drug product (produced in or derived from living organisms) may be demonstrated to be “biosimilar” if data show that, among other things, the product is “highly similar” to an already-approved biological product. The biosimilar product should retain at least the biologic function and treatment efficacy of the U.S. Food and Drug Agency-approved biological product. The biosimilar product can be formulated differently, however, from the approved biological product. A different formulation can provide improved stability and shelf storage of the biologic drug product, and can also improve the efficacy in treating a particular disease or condition. The different formulation can also improve other aspects of administration, such as a reduction in patient discomfort or other unwanted effects that a patient may experience upon administration of the approved biological product. Antibody molecules can be produced as a biosimilar nanoantibody and reformulated accordingly. There remains a need in the art for high quality antibody formulations, method of administration, and use thereof.

Caplacizumab is an injectable humanized bivalent anti-von Willebrand Factor (vWF) antibody fragment that consists of two identical building blocks, linked by three alanine residues. Caplacizumab is indicated for the treatment of adult patients with acquired thrombotic thrombocytopenic purpura, in combination with plasma exchange and immunosuppressive therapy. The administration of caplacizumab in combination with plasma exchange and immunosuppressive therapy has the disadvantage of patient discomfort and painful intravenous injection.

Therefore, there remains a need in the art for a stable formulation of caplacizumab solution for administration by inhalation using a soft mist inhaler or nebulizer.

### SUMMARY OF THE INVENTION

The present invention is directed to a stable formulation of the nanoantibody caplacizumab and a novel therapeutic strategy for the treatment of acquired thrombotic thrombocytopenic purpura by administering the caplacizumab using a soft mist inhaler or nebulizer. Therapeutic nanoantibodies for treating acquired TTP are formulated to form an aerosol using a soft mist inhaler. The aerosolized therapeutic nanoantibodies are locally delivered to the lungs by inhalation. The aim of pulmonary delivery of caplacizumab is to increase efficacy in treating acquired TTP by increasing lung deposition. This therapeutic strategy reduces the side effects of the drug because the nanoantibodies are absorbed through the alveoli and enter the blood circulatory system. The pulmonary delivery of a therapeutic nanoantibody through inhalation reduces the dosage of the therapeutic antibody compare to systematic IV administration and, thus, reduces the toxicity.

### BRIEF DESCRIPTION OF THE DRAWINGS

FIG. 1 shows a longitudinal section through an atomizer in the stressed state.

FIG. 2 shows the counter element of an atomizer.

### DETAILED DESCRIPTION OF THE INVENTION

The technical and nontechnical terminology used herein is for the purpose of describing particular embodiments only, and is not intended to be limiting of the invention. As used herein, the term “and/or” includes any and all combinations of one or more of the associated listed items. As used herein, the singular forms “a”, “an”, and “the” are intended to include the plural forms as well as the singular forms, unless the context clearly indicates otherwise. It will be further understood that the term “comprises” when used in this specification, specifies the presence of the stated features, steps, operations, elements, and/or components, but do not preclude the presence or addition of one or more other features, steps, operations, elements, components and/or groups thereof.

Unless otherwise defined, all terms (including technical and scientific terms) used herein have the same meaning as commonly understood by one having ordinary skill in the art to which the invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein the respiratory tract includes the oral and nasal-pharyngeal, tracheobronchial, and pulmonary regions. The pulmonary region is defined to include the upper and lower bronchi, bronchioles, terminal bronchioles, respiratory bronchioles, and alveoli.

In describing the invention, it will be understood that a number of formulations and steps are disclosed. Each of these has individual benefits and each can also be used in conjugation with one or more, or in some cases all, of the other disclosed techniques. Accordingly, for the sake of clarity, this description will refrain from repeating every possible combination of the individual steps in an unnecessary fashion. Nevertheless, the specification and claims should be read with an understanding that such combinations are entirely within the scope of invention and the claims.

The present disclosure is to be considered as an exemplification of the invention, and is not intended to limit the invention to the specific embodiments illustrated by the figures or description below.

The present invention describes a pharmaceutical formulation comprising an active therapeutic nanoantibody with other excipients that can be administered using a soft mist inhaler or nebulizer for the treatment of TTP. The formulations for use with the soft mist inhaler or nebulizer should meet standard quality guidelines. Therefore, one aim of the current invention is to provide a stable formulation containing a therapeutic nanoantibody in functional form with inactive ingredients in a solution, which meet the standard delivered dosage requirements needed to achieve optimum nebulization of the solution using a soft mist inhaler or nebulizer. In one aspect, the formulation maintains the activity of the active ingredient for the storage time indicated on the label. Another aspect is to provide a propellant-free solution containing a therapeutic nanoantibody and excipients, which can be nebulized under pressure using a soft mist inhaler or nebulizer. The amount of the composition delivered by the aerosol is reproducibly produced within a specified range.

Accordingly, the present invention relates to an inhalable therapeutic nanoantibody formulation containing caplacizumab as a major active molecule in combination with citric acid, polysorbate-80, sucrose, trisodium citrate dihydrate, or mixtures thereof. Preferably, the mixture is administered as an aerosol formed from a soft mist inhaler or nebulizer. The pharmaceutical formulations of the current invention are especially suitable for soft mist inhalation or nebulization, which have excellent lung deposition, typically up to 55-60%. Furthermore, administering liquid inhalation formulations of therapeutic nanoantibodies has other advantages compared to administering therapeutic nanoantibodies through an IV line, particularly for treating TPP.

In a further aspect, the present invention provides a solution for administration by inhalation using a soft mist inhaler or nebulizer comprising caplacizumab, water, sodium phosphate buffer, and an acid selected from hydrochloric acid, citric acid, or a mixture thereof, wherein the solution is substantially free of preservatives.

In yet another aspect, the present invention provides a nebulizer comprising a reservoir, wherein the reservoir contains one of the above-mentioned formulations.

The formulations of the present invention contain caplacizumab as an active agent. In one embodiment, the caplacizumab is caplacizumab-yhdp. The invention also relates to preparations in the form of an aqueous solution, which can be aerosolized, that contain as an active substance a biologically active macromolecule, particularly a therapeutic nanoantibody. The amount of caplacizumab will vary depending on the particular product, medical indication and patient. Typically, the amount of caplacizumab per inhalation ranges from about 0.01 mg to about 10 mg. Suitable doses include, but are not limited to, about 0.5, about 1, about 2, about 4, about 8, or about 12 mg. The volume of solution per inhalation typically ranges from about 0.5 ml to about 5 ml. In one embodiment, the volume of solution per inhalation ranges from about 0.5 ml to about 3.5 ml. This volume is preferably provided as a unit dose. In one embodiment, each dose is presented as a unit dose containing about 0.5 mg to about 12 mg of caplacizumab in about 0.5 ml to about 5 ml of solution. In one embodiment, each dose is presented as a unit dose containing about 0.5 mg to about 12 mg of caplacizumab in about 0.5 ml to about 3.5 ml of solution. In one embodiment, each dose is presented as a unit dose containing about 1 mg to about 4 mg of caplacizumab in about 0.5 ml to about 5 ml of solution. In one embodiment, each dose is presented as a unit dose containing about 1 mg to about 4 mg of caplacizumab in about 0.5 ml to about 3.5 ml of solution.

The soft mist inhalers nebulize a small amount of a liquid formulation containing the required dosage of the nanoantibodies into an aerosol that is suitable for therapeutic inhalation within a few seconds. A soft mist inhaler is particularly suitable for administering the liquid formulations disclosed in the current invention. A parameter of the aerosol, which is indicative of the aerosol quality, is the so-called inhalable proportion, which is defined herein as the proportion of the mist droplets with a measured median aero-dynamic diameter (MMAD) of less than about 15 μm. The inhalable proportion can be measured using an “Andersen Impactor”. For good protein absorption it is important to not only achieve aerosolization without any substantial loss of activity but also to generate an aerosol with a good inhalable proportion. Aerosols with an MMAD of less than about 10 μm are better suited to reaching the alveoli, where their chances of being absorbed are greater. The effectiveness of a soft mist inhaler (SMI) device can also be tested in an in vivo system. As an example of an in vivo test system, a protein-containing mist can be administered to a dog through a tracheal tube. Blood samples are taken at suitable time intervals and the protein level in the plasma are then measured by immunological or biological methods.

The pharmaceutical formulation according to present invention may be formulated using one or more physiologically acceptable carriers comprising excipients and auxiliaries known in the art. In one embodiment, the excipients and auxiliaries are selected from polysorbate-80, sucrose, and sodium citrate dihydrate.

In an embodiment, the formulations of the present invention also contain an acid. The acid lowers the pH of the formulation, providing chemical stability to the caplacizumab. The acid may be hydrochloric acid, citric acid, or a mixture of hydrochloric acid and citric acid.

The amount of acid required depends on the desired pH of the formulation. A nebulizer formulation having a pH ranging from about 2 to about 8 is acceptable to the patient. In one embodiment, the pH of the formulation ranges from about 5.5 to about 7. In one embodiment, the pH of the formulation ranges from about 6 to about 6.8. In one embodiment, the pH of the formulation ranges from about 6.4 to about 6.7. In one embodiment the pH of the formulation is about 6.5.

The formulation of the present invention can also contain polysorbate-80 as a surfactant. In one embodiment, the amount of polysorbate-80 ranges from about 0.05 mg/ml to about 0.15 mg/ml. In one embodiment, the amount of polysorbate-80 is about 0.1 mg/ml.

In an embodiment, the formulations of the present invention are sterile. Sterilization may be carried out by gamma irradiation or filtration. In an embodiment, the formulations are sterilized by filtration. The formulation may be a multi-dose or single dose formulation. In one embodiment, the formulation is a single-dose formulation. The formulation is typically provided in a container and hence the present invention also provides a container containing the formulation as defined herein.

In a further aspect, the present invention provides a nebulizer comprising a reservoir, wherein the reservoir contains an above described formulation. The nebulizer may be a jet nebulizer, a vibrating mesh nebulizer, an ultrasonic wave nebulizer, a soft-mist nebulizer, a high efficiency nebulizer, or a soft mist inhaler. In one embodiment, the nebulizer is a soft mist inhaler. In an embodiment, the formulation is advantageously administered to the patient using a soft mist inhaler or a metered dose inhaler.

A typical device for the propellant-free administration of a metered amount of a liquid pharmaceutical composition for soft mist inhalation is described in detail in, for example, US20190030268 entitled “inhalation atomizer comprising a blocking function and a counter”.

The pharmaceutical solution in the nebulizer is converted into aerosol destined for the lungs. The nebulizer uses high pressure to spray the pharmaceutical solution.

The inhalation device can be carried anywhere by the patient, since its cylindrical shape and handy size is less than about 8 cm to about 18 cm long, and about 2.5 cm to about 5 cm wide. The nebulizer sprays out a defined volume of the pharmaceutical formulation through small nozzles at high pressures, so as to produce an inhalable aerosol.

In one embodiment, the delivery device comprises an atomizer 1, a fluid 2, a vessel 3, a fluid compartment 4, a pressure generator 5, a holder 6, a drive spring 7, a delivering tube 9, a non-return valve 10, pressure room 11, a nozzle 12, a mouthpiece 13, an aerosol 14, an air inlet 15, an upper shell 16, and an inside part 17.

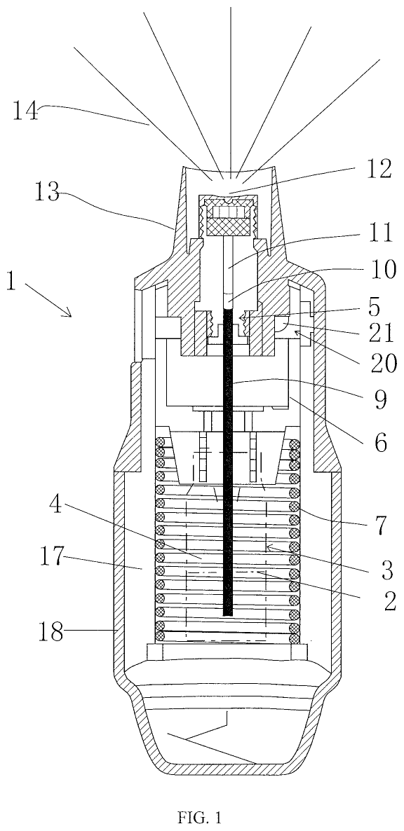

The inhalation atomizer 1 comprising the block function and the counter described above for spraying a medicament fluid 2 is depicted in the FIG. 1 in a stressed state. The atomizer 1 comprising the block function and the counter described above is preferably a portable inhaler and propellant-free.

FIG. 1 shows a longitudinal section through the atomizer in a stressed state.

For the typical atomizer 1 comprising the block function and the counter described above, an aerosol 14 that can be inhaled by a patient is generated through the atomization of the fluid 2, which is preferably formulated as a medicament liquid. The medicament is typically administered at least once a day, more specifically multiple times a day, preferably at predestined time gaps, according to how serious the illness affects the patient.

In an embodiment, the atomizer 1 described above has substitutable and insertable vessel 3, which contains the medicament fluid 2. Therefore, a reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the inhalation atomizer 1 comprising the block function and the counter described above is in the vessel 3 to provide, e.g., up to 200 doses. A classical vessel 3 has a volume of about 2 to about 10 ml. A pressure generator 5 in the atomizer 1 is used to deliver and atomize the fluid 2 in a predetermined dosage amount. Therefore, the fluid 2 can be released and sprayed in individual doses, specifically from 5 to 30 microliter.

In an embodiment, the atomizer 1 described above may have a pressure generator 5 and a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 could be separated from the atomizer 1 for substitution.

In an embodiment, when drive spring 7 is stressed in an axial direction, the delivering tube 9, the vessel 3 along with the holder 6 will be shifted downwards. Then the fluid 2 will be sucked into the pressure room 11 through delivering tube 9 and the non-return valve 10.

In one embodiment, after releasing the holder 6, the stress is eased. During this process, the delivering tube 9 and closed non-return valve 10 are shifted back upward by releasing the drive spring 7. Consequently, the fluid 2 is under pressure in the pressure room 11. Then the fluid 2 is pushed through the nozzle 12 and atomized into an aerosol 14 by the pressure. A patient could inhale the aerosol 14 through the mouthpiece 13, while the air is sucked into the mouthpiece 13 through air inlets 15.

The inhalation atomizer 1 described above has an upper shell 16 and an inside part 17, which can be rotated relative to the upper shell 16. A lower shell 18 is manually operable to attach onto the inside part 17. The lower shell 18 can be separated from the atomizer 1 so that the vessel 3 can be substituted and inserted.

In one embodiment, the inhalation atomizer 1 described above has the lower shell 18, which carries the inside part 17, being rotatable relative to the upper shell 16. As a result of rotation and engagement between the upper unit 17 and the holder 6, through a gear 20, the holder 6 is axially moved counter to the force of the drive spring 7 and the drive spring 7 is stressed.

In an embodiment, in the stressed state, the vessel 3 is shifted downwards and reaches to a final position, which is demonstrated in the FIG. 1. The drive spring 7 is stressed under this final position. Then the holder 6 is clasped. Therefore, the vessel 3 and the delivering tube 9 are prevented from moving upwards so that the drive spring 7 is stopped from easing.

In an embodiment, the atomizing process occurs after releasing the holder 6. The vessel 3, the delivering tube 9 and the holder 6 are shifted back by the drive spring 7 to the beginning position. This is referred to herein as major shifting in here. While the major shifting occurs, the non-return valve 10 is closed and the fluid 2 is under pressure in the pressure room 11 by the delivering tube 9, and fluid 2 is pushed out and atomized by the pressure.

In an embodiment, the inhalation atomizer 1 described above may have a clamping function. During clamping, the vessel 3 preferably performs a lifting shift for withdrawal of fluid 2 during the atomizing process. The gear 20 has sliding surfaces 21 on the upper shell 16 and/or on the holder 6, which makes holder 6 move axially when the holder 6 is rotated relative to the upper shell 16.

In an embodiment, the holder 6 is not blocked for too long and can perform the major shifting. Therefore, the fluid 2 is pushed out and atomized.

In an embodiment, when the holder 6 is in the clamping position, the sliding surfaces 21 move out of engagement. Then the gear 20 releases the holder 6 for the opposite shift axially.

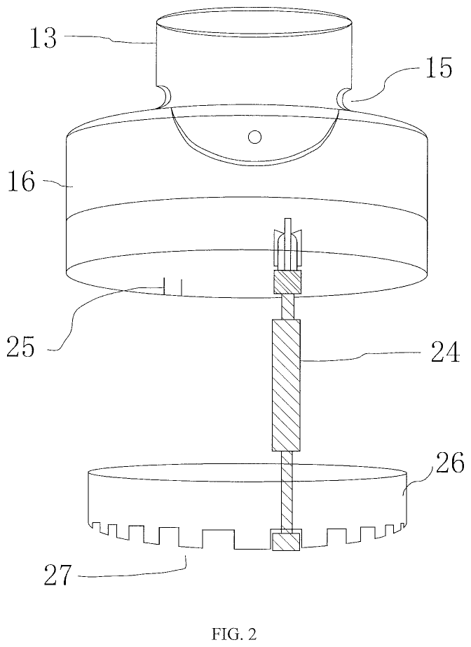

In an embodiment, the atomizer 1 preferably includes a counter element shown in FIG. 2. The counter element has a worm 24 and a counter ring 26. Preferably, the counter ring 26 is circular and has dentate part at the bottom. The worm 24 has upper and lower end gears. The upper end gear contacts with the upper shell 16. The upper shell 16 has inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear so as to result in a counting effect.

In an embodiment, the locking mechanism is realized mainly by two protrusions. Protrusion A is located on the outer wall of the lower unit of the inside part. Protrusion B is located on the inner wall of counter. The lower unit of the inside part is nested in the counter. The counter can rotate relative to the lower unit of the inside part. Because of the rotation of the counter, the number displayed on the counter can change as the actuation number increases, and can be observed by the patient. After each actuation, the number displayed on the counter changes. Once a predetermined number of actuations is achieved, Protrusion A and Protrusion B will encounter with each other and hence the counter will be prevented from further rotation. Therefore, the atomizer is blocked and stopped from further use. The number of actuations of the device can be counted by the counter.

The nebulizer described above is suitable for nebulizing the aerosol preparations according to the invention to form an aerosol suitable for inhalation. Nevertheless, the formulation according to the invention can also be nebulized using other inhalers apart from those described above, such as an ultrasonic vibrating mesh nebulizer or a compressed air nebulizer.

A typical ultrasonic vibrating mesh nebulizer is composed of a liquid reservoir with a piezo mesh disk mounted on one side of it and a piezo mesh driver circuit board with batteries. The piezo mesh disk consists of a stainless steel plate that has been perforated with thousands of precision-formed, laser-drilled holes, and surrounded by a piezoelectric material. The piezoelectric material vibrates at a very high rate of speed when it is driven by an analog signal of specific voltage, frequency, and waveform that is generated by the driver board. As a result of the rapid vibration, solution is drawn through the holes to form droplets of consistent size that are delivered at a low velocity for inhalation directly into the lungs.

With a typical compressed air nebulizer, an aerosol is generated by passing air flow in a nebulizer bowl. This forms a low-pressure zone that pulls up droplets through a feed tube from a solution or suspension of a drug in the nebulizer bowl, which in turn creates a stream of atomized droplets, which flow to the mouthpiece. Higher air flows lead to a decrease in particle size and an increase in output. A baffle in the nebulizer bowl is impacted by larger particles, retaining them and returning them to the solution in the nebulizer bowl to be re-atomized. There is considerable variation in the performance of nebulizers. In addition, nebulizers require a source of compressed air.

### EXAMPLES

### Example 1

An aqueous solution containing caplacizumab as a therapeutic nanoantibody for administration using a soft mist inhaler and/or nebulizer was prepared by combining the ingredients in table 1. The solution was adjusted to the pH with citric acid. Finally sterile water was added to provide a final volume of 10 ml.

TABLE 1
Formulation of sample I.

| Ingredients | Sample I | |
|---|---|---|
| Caplacizumab-yhdp | 100 | mg |
| Polysorbate-80 | 1 | mg |
| Sucrose | 620 | mg |
| Trisodium citrate dihydrate | 49.1 | mg |
| Anhydrous citric acid | 1.8 | mg |

| | |
|---|---|
| pH | 6.5 |
| Sterile water | To 10 ml |

### Example 2

An aqueous solution containing caplacizumab as a therapeutic nanoantibody for administration using a soft mist inhaler and/or nebulizer was prepared by combining the ingredients in table 2. The solution was adjusted to the pH with citric acid. Finally sterile water was added to provide a final volume of 10 ml.

TABLE 2
Formulation of sample II.

| Ingredients | Sample II | |
|---|---|---|
| Caplacizumab-yhdp | 200 | mg |
| Polysorbate-80 | 2 | mg |
| Sucrose | 1240 | mg |
| Trisodium citrate dihydrate | 98.2 | mg |
| Anhydrous citric acid | 3.6 | mg |

| | |
|---|---|
| pH | 6.5 |
| Sterile water | To 10 ml |

### Example 3

An aqueous solution containing caplacizumab as a therapeutic nanoantibody for administration using a soft mist inhaler and/or nebulizer was prepared by combining the ingredients in table 3. The solution was adjusted to the pH with sodium phosphate (monobasic, monohydrate) and sodium phosphate (dibasic). Finally sterile water was added to provide a final volume of 10 ml.

TABLE 3
Formulation of sample III.

| Ingredients | Sample III | |
|---|---|---|
| Caplacizumab-yhdp | 100 | mg |
| Polysorbate-20 | 2 | mg |
| Sodium phosphate (monobasic, | 30.2 | mg |
| monohydrate) | | |
| Sodium phosphate (dibasic) | 6.5 | mg |

| | |
|---|---|
| pH | 6.5 |
| Sterile water | To 10 ml |

### Example 4

An aqueous solution containing caplacizumab as a therapeutic nanoantibody for administration using a soft mist inhaler and/or nebulizer was prepared by combining the ingredients in table 4. The solution was adjusted to the pH with sodium phosphate (monobasic, monohydrate) and sodium phosphate (dibasic). Finally sterile water was added to provide a final volume of 10 ml.

TABLE 4
Formulation sample IV.

| Ingredients | Sample IV | |
|---|---|---|
| Caplacizumab-yhdp | 200 | mg |
| Polysorbate-20 | 4 | mg |
| Sodium phosphate (monobasic, | 60.4 | mg |
| monohydrate) | | |
| Sodium phosphate (dibasic) | 13 | mg |
| pH | 6.5 | |
| Sterile water | 10 | ml |

What is claimed is:

1. A liquid, propellant-free pharmaceutical formulation comprising: (A) caplacizumab in an amount ranging from about 0.01 mg/ml to about 100 mg/ml and (B) at least one pharmaceutically acceptable pH-adjusting agent.

2. The pharmaceutical formulation of claim 1, wherein the caplacizumab is caplacizumab-yhdp.

3. The pharmaceutical formulation of claim 1, further comprising a surfactant.

4. The pharmaceutical formulation of claim 3, wherein the surfactant is selected from the group consisting of polysorbate-80, polysorbate-20, and mixtures thereof.

5. The pharmaceutical formulation of claim 1, further comprising about 1 mg/ml to about 100 mg/ml sucrose, about 1 mg/ml to about 10 mg/ml of trisodium citrate dihydrate, and about 0.1 mg/ml to about 0.5 mg/ml anhydrous citric acid.

6. The pharmaceutical formulation of claim 1, comprising from about 50 mg/ml to about 500 mg/ml sucrose, about 5 mg/ml to about 15 mg/ml trisodium citrate dihydrate, and from about 0.1 mg/ml to about 0.5 mg/ml anhydrous citric acid.

7. The pharmaceutical formulation of claim 1, further comprising about 1 mg/ml to about 20 mg/ml sodium phosphate monobasic monohydrate and about 0.2 mg/ml to about 1.5 mg/ml sodium phosphate dibasic.

8. The pharmaceutical formulation of claim 1, wherein the pH adjusting agent is selected from the group consisting of citric acid, hydrochloric acid, and mixture thereof.

9. The pharmaceutical formulation of claim 1, wherein the pH adjusting agent is citric acid.

10. The pharmaceutical formulation of claim 1, wherein the pH adjusting agent is HCl.

11. The pharmaceutical formulation of claim 1, wherein the formulation is substantially free of preservatives and stabilizers.

12. The pharmaceutical formulation of claim 1, wherein the pH is in a range from about 6 to about 7.

13. The pharmaceutical formulation of claim 12, wherein the pH is in a range from about 6.4 to about 6.6.

14. A method of treating thrombotic thrombocytopenic purpura comprising administering the pharmaceutical formulation of claim 1 by inhalation.

15. A method of administering the formulation of claim 1, comprising nebulizing a defined amount of the pharmaceutical formulation with an inhaler by using pressure to force the pharmaceutical formulation through a nozzle to form an inhalable aerosol.

16. The method of claim 15, wherein the aerosol has a droplet size (d90) of less than about 15 μm.

17. The method of claim 16, wherein the droplet size (d90) is in a range from about 0.5 μm to about 15 μm.

18. The pharmaceutical formulation of claim 1 comprising:
an aqueous solution comprising:
caplacizumab-yhdp in an amount ranging from about 100 mg/10 ml to about 200 mg/10 ml,
polysorbate-80 in an amount ranging from about 1 mg/10 ml to about 2 mg/10 ml,
sucrose in an amount ranging from about 620 mg/10 ml to about 1240 mg/10 ml,
trisodium citrate dihydrate in an amount ranging from about 49.1 mg/10 ml to about 98.2 mg/10 ml, and
anhydrous citric acid in an amount ranging from about 1.8 mg/10 ml to about 3.6 mg/ml;
wherein the pH of the pharmaceutical formulation is about 6.5.

19. The pharmaceutical formulation of claim 1 comprising:
an aqueous solution comprising:
caplacizumab-yhdp in an amount ranging from about 100 mg/10 ml to about 200 mg/10 ml,
polysorbate-20 in an amount ranging from about 2 mg/10 ml to about 4 mg/10 ml,
sodium phosphate (monobasic, monohydrate) in an amount ranging from about 30.2 mg/10 ml to about 60.4 mg/10 ml,
sodium phosphate (dibasic) in an amount ranging from about 6.5 mg/10 ml to about 13 mg/10 ml;
wherein the pH of the pharmaceutical formulation is about 6.5
